(19) United States
(12) Patent Application Publication
(10) Pub. No.: US 2007/0239145 A1
(43) Pub. Date: Oct. 11, 2007

(54) SYSTEM AND METHOD TO ASSIST IN THE TREATMENT OF SKIN CONDITIONS

(76) Inventor: Raphael Laderman, San Francisco, CA (US)

Correspondence Address: SCHWEGMAN, LUNDBERG & WOESSNER, P.A., P.O. BOX 2938, MINNEAPOLIS, MN, 55402, US

(21) Appl. No.: 11/695,187

(22) Filed: Apr. 2, 2007

Related U.S. Application Data
(60) Provisional application No. 60/791,240, filed on Apr. 11, 2006.

(51) Int. Cl. A61B 18/18

(52) U.S. Cl. 606/9

(57) ABSTRACT

A system and method for treating skin includes detecting the response of defective skin to radiation at a level of radiation less than that which would damage healthy skin, detecting the response of healthy skin to radiation at a level of radiation less than that which would damage healthy skin, and adjusting radiation power as a function of the detected responses. The radiation may be applied to defective skin at the adjusted radiation power. In various embodiments, responses of skin to radiation may include electromagnetic absorption qualities of a patient's normal skin, electromagnetic absorption properties of the patient's skin in the area of the abnormal skin condition or blemish, and the differential between the two.

### RELATED APPLICATIONS

This application claims priority to U.S. Provisional Application Ser. No. 60/791,240 (entitled System and Method to Assist in the Treatment of Skin Conditions), filed Apr. 11, 2006) which is incorporated herein by reference.

### BACKGROUND

Many methods of treatment have been developed in the prior art for different types of skin problems. Some abnormal skin conditions or blemishes such as acial or leg telangiectasias, spider nevi, erythrosis interfollicularis, senile angiomas, rosacea, hemangiomas, port wine stains, solar lentigines and epidermal pigmented lesions, for example, can be treated by the use of Intense Pulsed Light (IPL) devices such as the Lumenis Quantum SR, the Quadra Q4 and other similar devices. Some of these conditions and other abnormal skin conditions or blemishes, including some tattoos, can be treated by the use of neodymium-doped yttrium aluminum garnet (Nd:YAG) lasers or other types of lasers. These methods of skin treatment using light or other electromagnetic radiation such as microwaves rely on utilizing frequencies which are more highly absorbed by the blemish or abnormal skin region to be treated as compared to the normal skin surrounding the blemish. This differential of light absorption allows blemishes or abnormal portions to be affected by the radiation while leaving the normal skin comparatively less affected.

One serious limitation of these methods is that they have been unable to optimally treat patients with darker skin or skin with more variable skin coloration. There is a need for a system and method that can operate effectively on many different skin tones.

### BRIEF DESCRIPTION OF THE DRAWINGS

FIG. 1 is a block diagram of a system for treating skin according to an example embodiment.

### DETAILED DESCRIPTION

In the following description, reference is made to the accompanying drawings that form a part hereof, and in which is shown by way of illustration specific embodiments which may be practiced. These embodiments are described in sufficient detail to enable those skilled in the art to practice the invention, and it is to be understood that other embodiments may be utilized and that structural, logical and electrical changes may be made without departing from the scope of the present invention. The following description of example embodiments is, therefore, not to be taken in a limited sense, and the scope of the present invention is defined by the appended claims.

The functions or algorithms described herein may be implemented in software or a combination of software and human implemented procedures in one embodiment. The software may consist of computer executable instructions stored on computer readable media such as memory or other type of storage devices. The term “computer readable media” is also used to represent any means by which the computer readable instructions may be received by the computer, such as by different forms of wired or wireless transmissions. Further, such functions correspond to modules, which are software, hardware, firmware or any combination thereof. Multiple functions may be performed in one or more modules as desired, and the embodiments described are merely examples. The software may be executed on a digital signal processor, ASIC, microprocessor, or other type of processor operating on a computer system, such as a personal computer, server or other computer system.

One serious limitation of prior methods of skin treatment is that they have been unable to optimally treat patients with darker skin or skin with more variable skin coloration. This may be due to one or more of the following factors: First, the electromagnetic absorptive properties of the patient's normal unblemished skin and/or the abnormal skin or blemished area are not measured sufficiently to determine the optimal or adequate electromagnetic frequencies and/or intensities to be used in the treatment. Second, there is not an adequate method of tuning the frequency and intensity of light, taking such measurement results into account so that the blemished or abnormal portions of skin are affected without affecting the normal skin of these patients. The result of these factors is that these methods tend to produce less than optimal results or are unusable for patients with darker skin or skin of varying color because they cause damage to normal, unblemished skin as well as to abnormal skin condition or blemished skin.

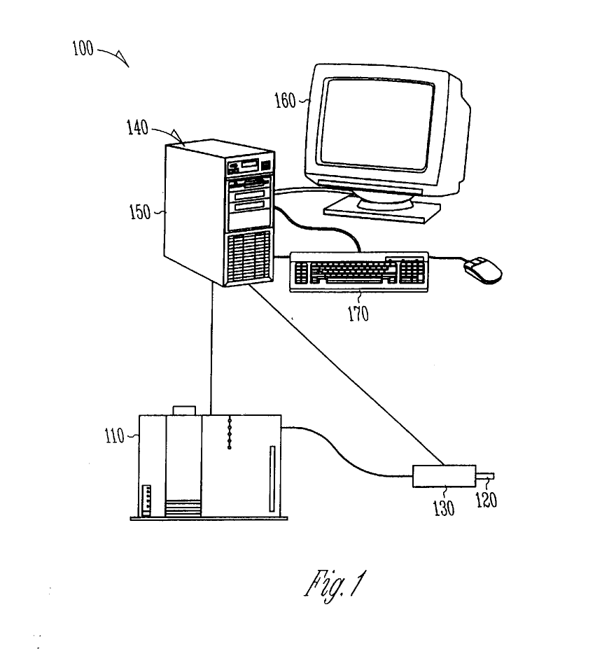

FIG. 1 illustrates an example embodiment of a system 100 for treating skin conditions. System 100 includes an electromagnetic radiation source 110 that is coupled to an emitter 120 that may be used to irradiate selected portions of skin to treat skin conditions. Some abnormal skin conditions or blemishes may include, but are note limited to acial or leg telangiectasias, spider nevi, erythrosis interfollicularis, senile angiomas, rosacea, hemangiomas, port wine stains, solar lentigines and epidermal pigmented lesions. Such skin conditions may be referred to generally as abnormal skin. A probe 130 is coupled to a computer system 140 and may be integrated into the emitter 120 or implemented as a separate unit. Computer system 140 is coupled to the radiation source 110 and optionally the probe 130 and emitter 120 to provide for analysis and control. Software in the computer system 140 provides functions for viewing and controlling settings, and optionally providing further analysis of data from the probe 130. A display 160 and various user input mechanisms 170 such as a keyboard, mouse, touchpad, joystick, etc., may be used to facilitate interaction with the computer system 140 and software 150.

Electromagnetic radiation source 110 is a means which is capable of producing one or a plurality of frequencies of electromagnetic radiation at one or a plurality of power levels sufficient for treating one or a plurality of types of skin conditions or blemishes. Several different adjustable radiation sources are commercially available. In different embodiments, these frequencies may be in one or a plurality of the visible, infra-red, ultra-violet or microwave electromagnetic wavelengths. In some embodiments, the radiation source may be one or a plurality of: lasers; light-emitting diodes; incandescent lamps; fluorescent lamps; microwave generators; or any other electromagnetic radiation source.

Emitter 120 is a means which is capable of delivering the radiation produced by the radiation source to the patient's skin. In different embodiments the emitter may utilize one or a plurality of: fiber optics; light guides; mirrors; microwave guides; or any other means of guiding the radiation to specific places on the patient's skin. The size of the area of skin to which the radiation is delivered may also differ in different embodiments. In some embodiments only a small pinpoint of radiation is delivered to the skin at any one time, while in other embodiments a larger area of radiation is delivered to the skin. In some embodiments the frequency or frequencies and power or powers of radiation delivered by the emitter are substantially the same across the entire area of delivery of radiation from the emitter 120 to the patient's skin. In other embodiments, one or a plurality of the frequencies and/or power levels of radiation may vary across the area of delivery of radiation from the emitter to the patient's skin. In some of these latter embodiments, the frequencies and/or power levels delivered to specific points or areas of the patient's skin may be specifically controlled. In some embodiments, the radiation beam is stationary, while in other embodiments it may be scanned across the surface of the skin or otherwise may be mobile. In some embodiments, one or a plurality of frequencies and/or power levels of radiation are delivered at a time, while in other embodiments, one or a plurality of frequencies and/or power levels of radiation are delivered sequentially. Some embodiments include a plurality of emitters of the same or of different types, while other embodiments do not include an emitter.

Probe 130 typically contains measuring means which may include one or a plurality of radiation-detectors or cameras, which are capable of measuring selected properties of the patient's skin. In some embodiments, an emitter is utilized to provide electromagnetic radiation for the purpose of illuminating the skin such that the detectors or cameras can collect and measure reflected radiation from the patient's skin. In other embodiments, a separate light source or radiation source is utilized by the probe instead of or in addition to the emitter. In some embodiments, the measuring means is located in a separate unit, and the radiation to be measured is conducted to that unit via one or a plurality of: fiber optics; light guides; mirrors; microwave guides; or any other means of guiding the radiation. When the probe 130 is triggered, the skin under the probe is illuminated by electromagnetic radiation of an intensity or power level less than that required to produce substantial damage to healthy/normal skin. The radiation detectors or cameras detect the reflection of this radiation by the skin and, in some embodiments, the emission of radiation by the skin in response to the illumination, as in the case of infra-red radiation emitted due to heating In some embodiments, several different frequencies of radiation may be utilized either sequentially or simultaneously in order to determine the properties of the skin. Some embodiments may include a plurality of probes of the same or different properties and/or sizes.

Computer system 140 consists of a processor, memory and non-volatile storage means, and is capable of obtaining input from the probe 130 and from the user input mechanisms 170, and displaying information on the display 160 or otherwise storing the information in one or more computer readable media. In one example embodiment, the computer system 140 is also capable of controlling the variable frequency electromagnetic radiation source 110 with respect to instructing it as to the intensity of radiation to produce and, in some embodiments, the duration of radiation pulses and, in some embodiments, the frequency or frequencies of electromagnetic radiation to produce.

Software 150 is machine-executable code stored, in one embodiment, in the non-volatile storage of the computer system, and which is capable of implementing the analysis and operational functions described herein.

Display 160 is an output means which gives feedback to the practitioner who is operating the device. Any type of display may be used that provide visual information to a user. Further, display 160 may comprise an audio device for providing audible verbal or acoustic information.

User input mechanisms 170 provide a way for the practitioner to enter information and select operational options. In one embodiment, this comprises a keyboard and mouse. There are other means of user input in a various other embodiments, which may include buttons on the probe and/or emitter, voice activated mechanisms or others.

In operation of one example embodiment, the practitioner may examine the patient to determine what type of abnormal skin condition she has which will be treated. The practitioner then interacts with the software running on the computer system by means of the user input mechanism keyboard and mouse and display console and enters this information or selects it from a menu of skin conditions.

The practitioner may then perform a pre-emission probe operation as follows. He first places the probe on a normal or healthy area of the patient's skin and triggers the probe. He then places the probe on an abnormal portion of the patient's skin corresponding to an area which is affected by the skin condition or blemish to be treated and again triggers the probe. In some embodiments, the probe measures a small pinpoint area of skin surface, allowing the practitioner to precisely and separately measure healthy or normal skin areas and abnormal condition or blemish areas. In other embodiments, the probe covers a larger area, and may be placed over an area of skin which contains both healthy/normal skin areas and abnormal-condition/blemish areas. In these embodiments, the probe measures a larger area of skin and the computer system and software detect the differential between the healthy/normal skin areas and the abnormal-condition/blemish areas without requiring the practitioner to reposition the probe. This may be done using known image analysis software, by scanning, or through other means. Other embodiments utilize the probe only or primarily to measure healthy skin.

When the probe operations have been completed the computer system and software utilize the information collected by the probe to determine one or a plurality of the following parameters: electromagnetic absorption qualities of the patient's normal skin; electromagnetic absorption properties of the patient's skin in the area of the abnormal skin condition or blemish; and the differential between the two.

Utilizing this information, the computer system and software can determine which frequencies, magnitudes and exposure times of electromagnetic radiation can be utilized to transfer energy to the patient's abnormal skin condition area or blemish while leaving the normal skin area relatively or acceptably unaffected. This may be done using one or more lookup tables corresponding to different skin conditions that may be indexed by the various measured or derived skin properties. The table or tables may be derived empirically, such as by experimentation, or calculated by one skilled in the art. It may be algorithmic in further embodiments, such as an expert system or neural network for example.

Some embodiments contain a computer system and software capable of utilizing the probe information to diagnose the type of skin abnormalities or blemishes present. In some of these embodiments, the initial step of the practitioner specifying via the user input mechanism the type of skin condition or blemish to be treated is not required. Some embodiments which perform diagnoses can utilize the diagnostic results to further determine more optimal parameters of electromagnetic radiation frequency or frequencies and/or power or powers to be used to treat the diagnosed skin abnormalities or blemishes.

In one embodiment, the computer system and software automatically configure the radiation source with optimal or adequate parameters such that the desired frequency or frequencies and power or powers of electromagnetic radiation are delivered to the patient's skin when requested by the practitioner. In other embodiments, the determinations made by the computer system and software utilizing the probe information are either transmitted to a separate radiation source, are stored in removable digital storage device, or are displayed on an display, printer or other output device. In some embodiments, the probe information is used to calculate an optimal or maximal recommended radiation exposure in order to induce a desired effect on healthy skin. An example of one such embodiment is a probe connected to a computer system with software which performs an analysis of the patient's skin and calculates optimal or maximal durations of sun exposure such that a tan is produced while minimizing ill effects such as sunburn. Some such embodiments can measure the sunlight as well as the patient's skin, and can utlize this combined information to more exactly calculate optimal or maximal durations of sun exposure. Another related embodiment is a tanning bed in which the patient's skin is measured, and this information is utilized to calculate the duration and intensity of ultraviolet radiation exposure such that a tan is produced while minimizing ill effects such as sunburn.

To perform the next step of the treatment, the Practitioner can utilize the radiation source and emitter to treat the abnormal skin condition or blemish by performing an emission operation. To perform the emission operation step in one embodiment, the practitioner places the emitter in contact with the patient's skin covering all or part of the area to be treated (ie: the area containing all or part of the abnormal skin condition or blemish), and triggers the radiation source utilizing a user input method which in some embodiments is one or a plurality of: a foot switch; a trigger on the emitter; a button pressed using the hand not holding the emitter; or another input device. In response to this input, the radiation source produces one or a plurality of pulses of one or a plurality of frequencies of electromagnetic radiation at one or a plurality of power levels.

The result of the pulse or pulses in some embodiments is measured again by a probe operation to help determine if they have been effective in treating the abnormal skin condition or blemish.

In some embodiments, the probe and emitter are combined into one probe/emitter unit such that a probe operation and an emission operation may be performed without repositioning the probe/emitter. Some of these embodiments may additionally utilize a separate probe for performing fine measurements.

In some embodiments which utilize a probe/emitter, the probe/emitter may conduct other types of probe operations. A first type of probe operation, as described above, is a pre-emission probe operation. Another type of probe operation which may be performed in some embodiments is a post-emission probe operation. This type of probe operation may measure the infrared energy and/or other electromagnetic energy emitted by the skin after an emission operation, in order to determine the amount or relative quantity of electromagnetic energy which was absorbed and/or reflected by the skin. Some embodiments may determine the differential energy absorbed and/or reflected by healthy/unblemished skin and abnormal/blemished skin areas and can utilize this information as feedback in order to tune the frequencies and/or power of electromagnetic radiation to be utilized in subsequent pulses. Other Embodiments merely measure the total energy absorbed and/or reflected by the skin area beneath the probe/emitter's sensing region. In some embodiments, one or a plurality of lower-intensity pulses may be utilized in a tuning process. In these embodiments, the electromagnetic radiation absorption and/or reflectance information obtained by the probe after these initial lower-intensity emissions is utilized by the computer system and software in order to tune the frequencies and/or power of electromagnetic radiation to be utilized in subsequent higher-intensity pulses. One objective of performing initial lower-intensity pulses, tuning, and then performing higher intensity pulses is to further minimize damage to healthy/unblemished skin and/or to further maximize electromagnetic radiation absorption by abnormal/blemished areas of skin.

In further embodiments, reflected radiation can be measured during treatment, and the treatment radiation being applied simultaneously can be varied in power, frequency or duration as a result of the measurements. This can be simultaneous or sequential (for instance, a pulse of radiation is applied, and then the reflected infrared is measured, changing the parameters for the next pulse). In still further embodiments, during treatment there can be more than one frequency and/or power of radiation applied. One or more frequency or power can be for treatment, and simultaneously or sequentially to the treatment radiation, there can be other frequencies and/or powers of radiation applied in order to perform measurements.

This discussion of embodiments has focused on systems which utilize a variable frequency electromagnetic radiation source, however other embodiments may utilize one or a plurality of fixed-frequency electromagnetic radiation sources or may not directly utilize any radiation source. Some embodiments which utilize or are used in conjunction with one or a plurality of fixed-frequency electromagnetic radiation sources may be utilized by a practitioner to help determine whether the patient is a good candidate for the frequencies produced by the given radiation source or sources. Some of these embodiments may also be able to suggest power outputs or radiation intensities or to control the power output or radiation intensity of one or a plurality of fixed-frequency electromagnetic radiation sources. Some embodiments may be utilized by a practitioner to help determine which one or plurality of the available fixed-frequency electromagnetic radiation sources are most appropriate for treating the patient and her abnormal skin condition or blemish, or whether the patient is a good candidate for the frequencies produced by any of the available radiation sources.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated.

Accordingly, one or more of the various embodiments may have one or more of the following advantages. It allows practitioners to measure the patient's skin and to determine optimal or adequate frequency or frequencies and/or intensity or intensities of electromagnetic radiation which will allow the delivery to the patient's skin of radiation which is preferentially absorbed by the blemish or other abnormal condition to be treated.

It allows practitioners to measure the patient's skin to determine optimal or adequate frequency or frequencies and/or intensity or intensities of electromagnetic radiation which will allow the delivery to the patient's skin of radiation which is absorbed to a lesser degree by the normal healthy portions of skin.

It may help avoid the problem of being unable to determine whether results will be adequate on patients with a variety of skin types. It avoids the problem of being unable to perform treatments on patients with some skin types. It reduces damage to healthy or unblemished skin which can be caused by the treatment.

The Abstract is provided to comply with 37 C.F.R. §1.72(b) to allow the reader to quickly ascertain the nature and gist of the technical disclosure. The Abstract is submitted with the understanding that it will not be used to interpret or limit the scope or meaning of the claims.

1. A method of treating skin comprising:
detecting a radiation response of defective skin to radiation at a level of radiation less than that which would damage healthy skin;
adjusting radiation power as a function of the response detected; and
applying radiation to treat defective skin at the adjusted radiation power.

2. The method of claim 1 wherein several different frequencies of radiation power are adjusted and applied, which may be different frequencies for detecting and applying.

3. The method of claim 2 wherein the different frequencies of radiation are applied sequentially or at least partially simultaneously.

4. The method of claim 1 wherein the radiation comprises laser light.

5. The method of claim 1 wherein the radiation is applied in pulses.

6. The method of claim 1 wherein the response of defective skin to radiation comprises electromagnetic absorption qualities of the defective skin.

7. The method of claim 1 wherein the detected radiation may have a frequency different from the radiation causing the skin to respond.

8. The method of claim 1 wherein the response of skin to radiation is detected responsive to the applied radiation, and the applied radiation may be modified as a function of such detection.

9. A method of treating skin comprising:
detecting the response of defective skin to radiation at a level of radiation less than that which would damage healthy skin;
detecting the response of healthy skin to radiation at a level of radiation less than that which would damage healthy skin;
adjusting radiation power as a function of the detected responses; and
applying radiation to treat defective skin at the adjusted radiation power.

10. The method of claim 9 wherein the detected responses comprise the following parameters:
electromagnetic absorption qualities of a patient's normal skin;
electromagnetic absorption properties of the patient's skin in the area of the abnormal skin condition or blemish; and
the differential between the two.

11. The method of claim 10 wherein several different frequencies of radiation power are adjusted and applied sequentially or at least partially simultaneously.

12. The method of claim 11 wherein the radiation is applied in pulses.

13. The method of claim 9 wherein the response of skin to radiation comprises electromagnetic absorption qualities of the defective skin.

14. The method of claim 9 and further comprising:
determining which frequencies of electromagnetic radiation can be utilized to transfer energy to abnormal skin while leaving the normal skin area relatively or acceptably unaffected.

15. The method of claim 9 and further comprising:
utilizing the detected responses of skin to diagnose the type of skin abnormalities present.

16. A system comprising:
means for detecting the response of defective skin to radiation at a level of radiation less than that which would damage healthy skin and detecting the response of healthy skin to radiation at a level of radiation less than that which would damage healthy skin;
means for adjusting radiation power as a function of the detected responses; and
means for applying radiation to treat defective skin at the adjusted radiation power.

17. A system for treating skin, the system comprising:
a probe that detects the response of defective skin to radiation at a level of radiation less than that which would damage healthy skin and detecting the response of healthy skin to radiation at a level of radiation less than that which would damage healthy skin;
a radiation source that applies radiation to defective skin; and
a computer system coupled to the probe and to the radiation source, wherein the computer system determines radiation power level and one or more frequencies a function of the detected responses and sets corresponding parameters of the radiation source.

18. A method comprising:
applying radiation to healthy and defective skin areas on a body;
detecting the response of the defective skin to radiation at a level of radiation less than that which would damage healthy skin;
detecting the response of healthy skin to radiation at a level of radiation less than that which would damage healthy skin;
adjusting radiation power as a function of the detected responses; and
applying radiation to treat defective skin at the adjusted radiation power.

19. The method of claim 18 wherein the response is at least one of reflected radiation and radiated radiation at frequencies which may be different than the applied radiation.

20. The method of claim 18 wherein the response of skin may be detected during application of radiation to defective skin at the adjusted radiation power to further adjust the radiation power.
